(19) United States
(12) Patent Application Publication
(10) Pub. No.: US 2008/0015613 A1
(43) Pub. Date: Jan. 17, 2008

(54) System and method for endoscopic treatment of tissue

(75) Inventors: Zahid A. Saeed, Cincinnati, OH (US); Nib Soehendra, Henstedt-Ulzburg (DE); John Mugan, Moycullen (IE)

Correspondence Address: BRINKS HOFER GILSON & LIONE/CHICAGO/COOK, PO BOX 10395, CHICAGO, IL, 60610, US

(73) Assignees: Wilson-Cook Medical Inc., Winston-Salem, NC (US); Cook Ireland Limited, Limerick (IE)

(21) Appl. No.: 11/725,601

(22) Filed: Mar. 19, 2007

Related U.S. Application Data
(63) Continuation-in-part of application No. 11/127,554, filed on May 12, 2005.
(60) Provisional application No. 60/571,279, filed on May 13, 2004.
(60) Provisional application No. 60/787,759, filed on Mar. 31, 2006.

(51) Int. Cl. A61B 17/10

(52) U.S. Cl. 606/139; 600/104

(57) ABSTRACT

A system and method for endoscopic treatment of tissue is disclosed. An endoscope has an operating channel. A mounting component has a first threading channel that fits into the operating channel of the endoscope. A ligating barrel fits onto a distal end of the endoscope. An elongate medical instrument is inserted through the first threading channel, the operating channel, and the ligating barrel to treat the tissue.

### CROSS-REFERENCE TO RELATED APPLICATIONS

This application is a continuation-in-part of U.S. Non-Provisional application Ser. No. 11/127,554, filed May 12, 2005, which claims the benefit of U.S. Provisional Application Ser. No. 60/571,279, filed May 13, 2004, both of which are entitled “System and Method For Endoscopic Treatment of Tissue”, the entire contents of which are incorporated herein by reference.

This application also claims the benefit of U.S. Provisional Application Ser. No. 60/787,759, filed Mar. 31, 2006, entitled “System and Method For Endoscopic Treatment of Tissue”, the entire contents of which are incorporated herein by reference.

### TECHNICAL FIELD

This system is related to medical devices and more particularly, to endoscopic treatment of tissue.

### BACKGROUND OF THE INVENTION

The treatment of tissue encompasses a variety of techniques such as electrocauterization, heat therapy, resection (removal of tissue), and sclerotherapy (the injection of medicine into target tissue). These treatment techniques usually involve the passing of medical instruments through the operating channel of the endoscope. The endoscope permits minimally invasive access, as well as visualization and suction aids.

Another technique that frequently utilizes the operating channel of the endoscope is ligation, which involves applying a band or ligature around a vessel or portion of tissue, thereby cutting off blood or fluid flow and causing the tissue to necrose and separate from adjacent healthy tissue. Ligation is widely used to treat a number of medical tissue conditions, including, but not limited to, hemorrhoids, polyps, ballooning varices, and other types of lesions, including those that are cancerous. Typically, ligators are also used with a suction or vacuum means to draw the tissue into the distal tip, whereby the band is deployed over the base of the diseased tissue to cut off blood flow. The ligating device is typically activated by retracting a line (string, wire, or cable) that is attached to the ligator at the distal end of an endoscope and is threaded through the operating channel of the endoscope to the proximal end of the instrument. The ligator can be activated by mechanically pulling the activating line by means of a hand-operated reel or trigger, or a motor drive mechanism. Various other ligating devices use cooperating inner and outer members that slide the individual bands by pushing or pulling them from the tip of the inner or outer member, the bands being preloaded onto the inner or outer member prior to deployment.

To prevent having to withdraw the instrument from the patient, reload, and reintroduce it for treating additional tissue or vessels, devices have been developed capable of sequentially delivering multiple bands that are preloaded, thus shortening the procedure time and improving patient comfort. Multiple band ligating devices include designs that individually tether or otherwise secure the bands to the dispenser and then release them sequentially as needed, often by use of one or more strings extending to the proximal end.

It is often desirable to combine another endoscopic procedure, such as sclerotherapy or tissue removal with a surgical snare, with ligation. However, while the operating channel of the endoscope is often large enough to accommodate more than just an activating line from a ligator, combining the medical instruments necessary for the second procedure with the ligator can be problematic. Thus, there is a need for a ligating device that can be combined with other medical instruments in endoscopic procedures.

### BRIEF SUMMARY

The present invention provides a system and method for endoscopic treatment of tissue. In particular, a system is provided for use an endoscope having an operating channel. The system includes a ligating system having an activating component and a ligating barrel. The system further includes a medical instrument having an elongate shaft and an operating member disposed near the distal end thereof. The activating component includes a mounting component having a first threading channel that fits into the working channel of the endoscope. In one preferred aspect of the invention, the first threading channel of the mounting component has a diameter of at least 2.5 millimeters, and more preferably a diameter of about 3.2 millimeters. The ligating barrel fits onto a distal end of the endoscope and is operably connected to the activating component. The shaft of the medical instrument is disposed through the first threading channel, the operating channel, and the ligating barrel so as to position the operating member near the tissue to be treated.

In another aspect, the present invention provides a system useful for convenient endoscopic resection of tissue. The system includes an endoscope having an operating channel, and a ligating barrel extending from the channel. The ligating barrel includes at least one and desirably multiple ligating bands disposed thereon and deployable therefrom. An elongated tissue resection device extends through the working channel and is effective to resect tissue captured by the bands. In certain embodiments the tissue resection device is an electrosurgical snare.

In another aspect the invention provides a method for endoscopically resecting tissue. The method includes advancing an endoscope into a body passageway of a patient. The endoscope has an operating channel and a ligating barrel extending from the channel. The patient's tissue is drawn into the ligating barrel and a ligating band is deployed to form a ligated tissue mass or pseudo-polyp. An elongated tissue resecting device, such as an electrosurgical snare, is advanced through the operating channel of the endoscope, and is used to resect the ligated tissue mass.

These and other features of the invention will become apparent upon review of the following detailed description of the presently preferred embodiments of the invention, taken into conjunction with the appended figures.

### BRIEF DESCRIPTION OF THE DRAWINGS

FIG. 1 is a longitudinal fragmented view of an endoscope with a ligating system.

FIG. 2 is a top plan view of the activating mechanism of the ligating system of FIG. 1.

FIGS. 3A-3C are views of a mounting component of the activating mechanism of FIG. 2.

FIGS. 4A-4E are views of a base of the activating mechanism of FIG. 2.

FIG. 5 is a view of a spool of the activating mechanism of FIG. 2.

FIGS. 6-7 are views of the activating mechanism of FIG. 2 with the incorporation of a surgical snaring system in the present invention.

FIGS. 8A-8B are views of a disengaged mode of operation of the activating mechanism of FIG. 2 wherein knob 60 is free to rotate in either direction (A or B).

FIGS. 9A-9B are views of an engaged mode of operation of the activating mechanism of FIG. 2 wherein knob 60 is only free to rotate in a single direction (A).

FIG. 10 is a view of the activating mechanism of FIG. 2 prior to insertion into the operating channel of the endoscope of FIG. 1.

FIG. 11 is a view of the activating mechanism of FIG. 2 after insertion into the operating channel of the endoscope of FIG. 1.

FIG. 12 is a cross-sectional view of a ligating barrel with the incorporation of the surgical snaring system in another embodiment of the present invention.

### DETAILED DESCRIPTION OF THE DRAWINGS AND THE PRESENTLY PREFERRED EMBODIMENTS

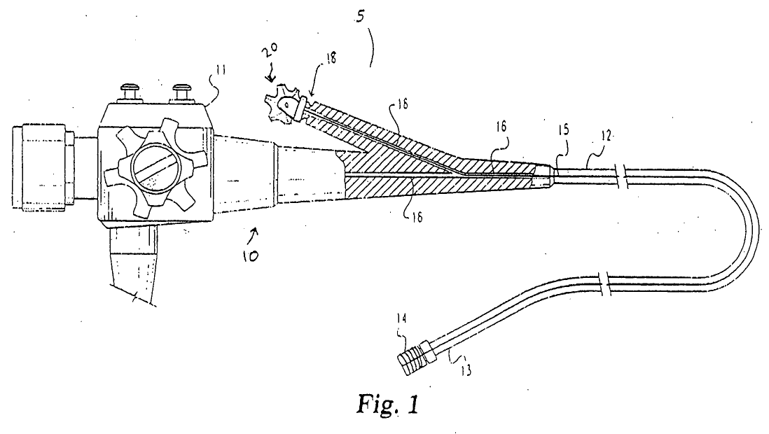

Referring now to the drawings, there is shown in FIG. 1 a ligating system 5 with endoscope 10. Ligating system 5 has a ligating barrel 14 attached to the distal end of endoscope 10. Ligating barrel 14 is generally shown in FIG. 1. A more detailed description of barrel 14 is shown in U.S. Pat. No. 5,624,453, which disclosure is hereby incorporated by reference. Endoscope 10 is a conventional endoscope with an operating control portion 11, a flexible section 12, and a distal end portion 13.

Ligating barrel 14 is located at distal end portion 13 of endoscope 10 and includes an activation line 15. Endoscope 10 also includes operating channel 16 which extends through endoscope 10 from ligating barrel 14 to both operating control portion 11 and to proximal opening 18. Activation line 15 is threaded from ligating barrel 14 through operating channel 16 and exits through proximal opening 18. Barrel 14 is preferably of a hard plastic clear polycarbonate for maximum durability and visibility.

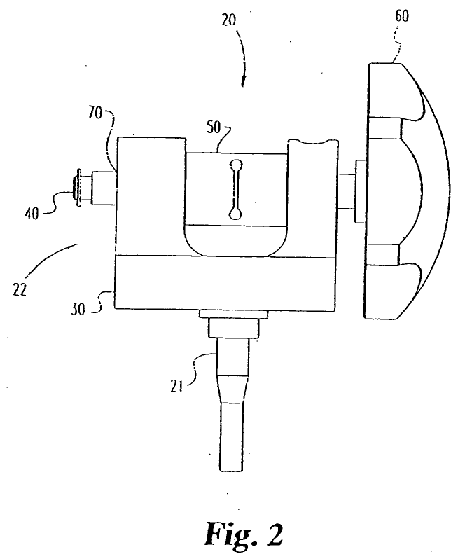

FIG. 2 is a top plan view of an activating mechanism 20 for ligating system 5. Activating mechanism 20 includes mounting component 21 and activating component 22. Activating component 22 includes base 30, drive pin 40, spool 50, knob 60 and roller clutch 70. Mounting component 21 attaches to activating component 22 and is used to mount activating mechanism 20 to endoscope 10 at proximal opening 18.

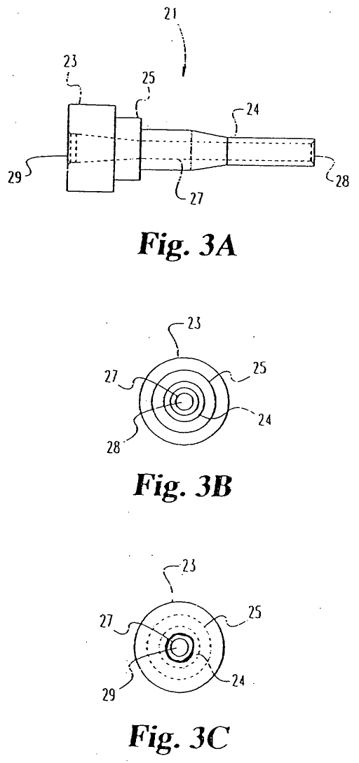

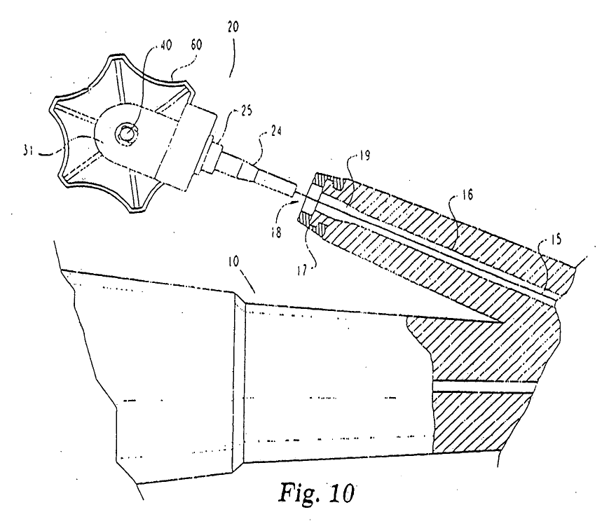

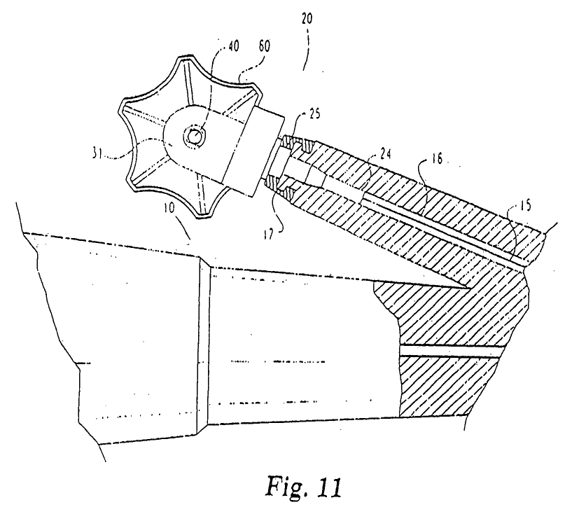

FIG. 3A is a view of mounting component 21. Mounting component 21, which is preferably formed of resilient material such as a plastic or a polyurethane, and more preferably formed of a strong material such as aluminum, includes coupling portion 23, friction-fit portion 24, and outer sealing portion 25. Coupling portion 23 is adapted to fixedly attach to activating component 22. Upon insertion into operating channel 16 through proximal opening 18, friction-fit portion 24 is sized to form a friction fit within operating channel 16. Where endoscope 10 is provided with a sealing member 17 (as shown in FIGS. 10 and 11), outer sealing portion 25 is adapted to seat in and seal with sealing member 17.

Mounting component 21 also provides first threading channel 27 which extends through mounting component 21. First opening 28 of first threading channel 27 is shown in FIG. 3B and second opening 29 of first threading channel 27 is shown in FIG. 3C. After mounting component 21 has been inserted into operating channel 16, activation line 15 can be threaded into first opening 28 and out of second opening 29 for connection to activating component 22.

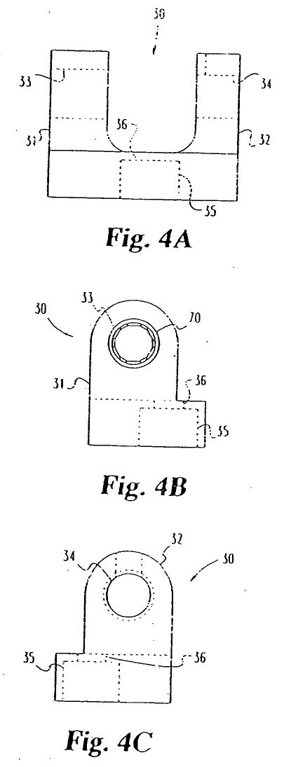

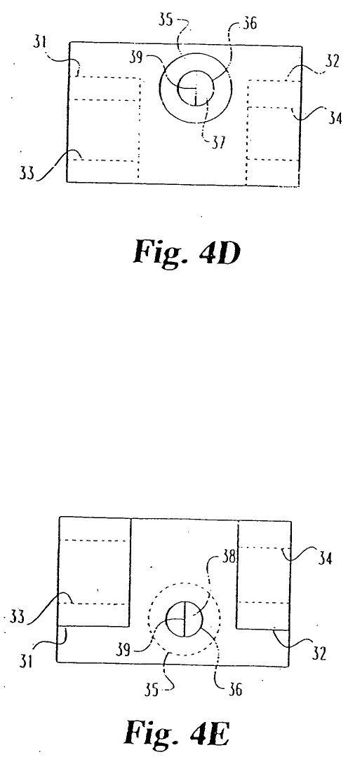

FIG. 4A is a frontal view of base 30. Base 30 includes first arm 31 and second arm 32. As shown in FIG. 4B, first arm 31 provides first receiving opening 33 and as shown in FIG. 4C, second arm 32 provides second receiving opening 34. Base 30 further includes recess 35 located on the bottom side portion of base 30 and second threading channel 36 which extends through recess 35 to the top side of base 30. FIG. 4D is a view of the bottom side of base 30, and further shows recess 35 and first opening 37 of second threading channel 36. FIG. 4E is a view of the top side of base 30, showing the top side of first arm 31 and second arm 32, and second opening 38 of second threading channel 36 with inner sealing member 39 located therein. When activation line 15 is threaded through mounting component 21, it is further threaded into first opening 37, through inner sealing member 39, and out of second opening 38 of base portion 30.

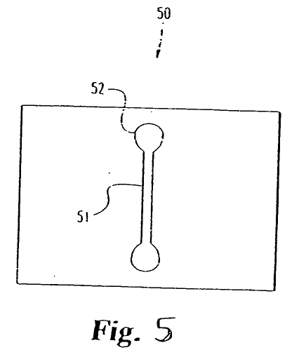

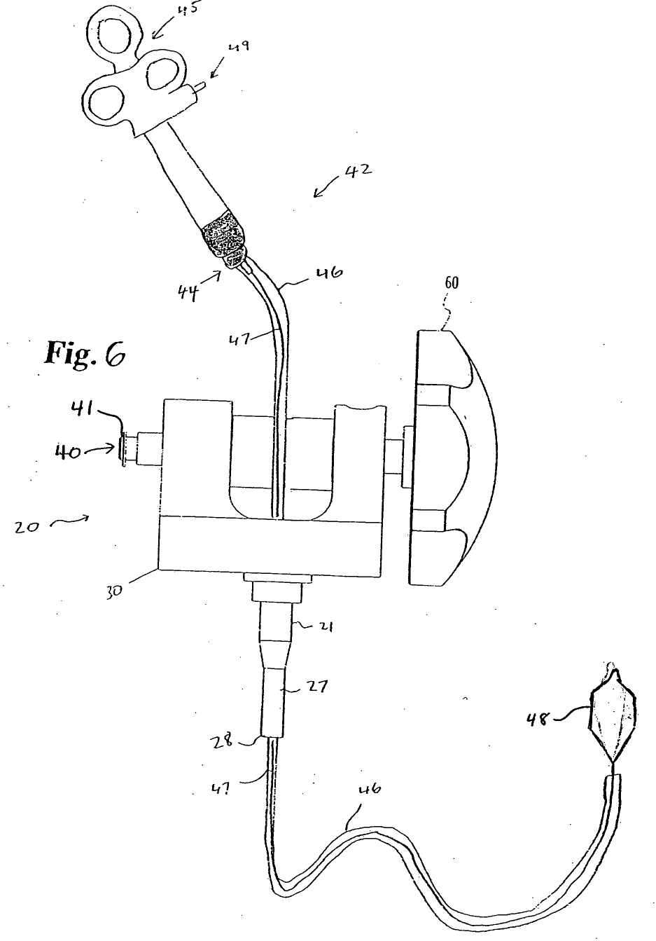

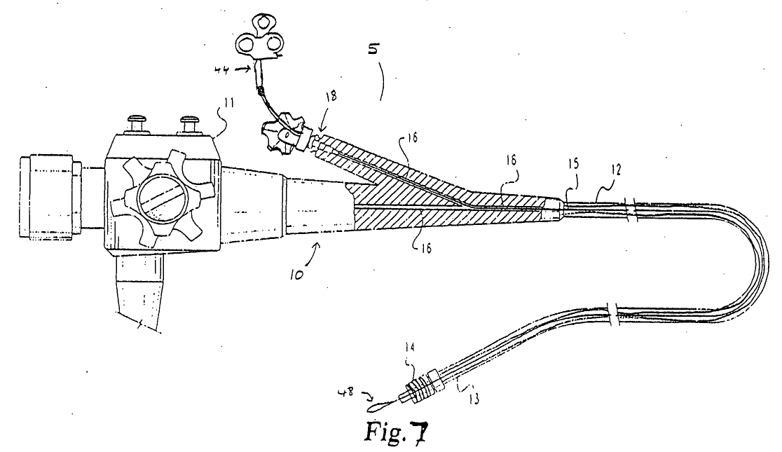

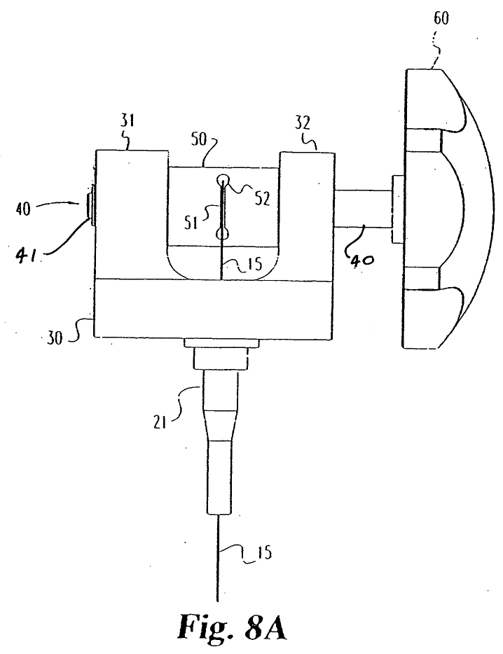

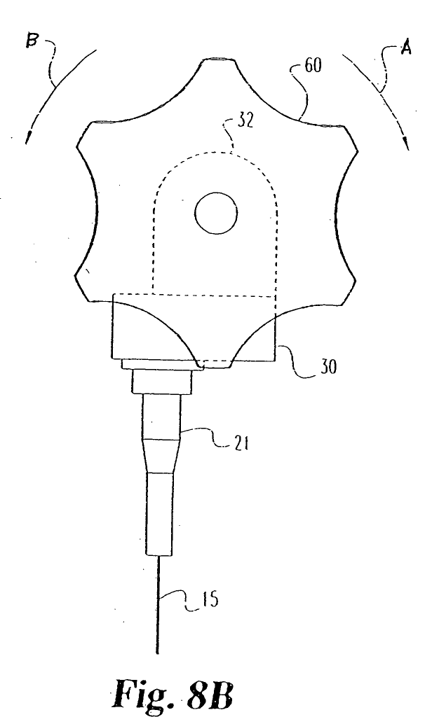

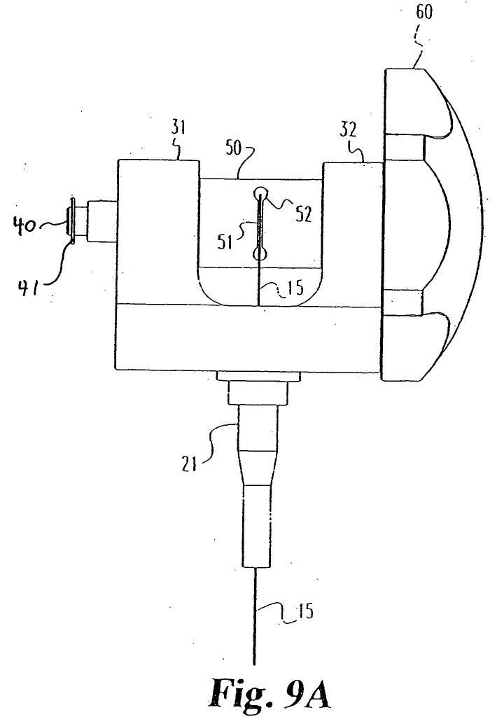

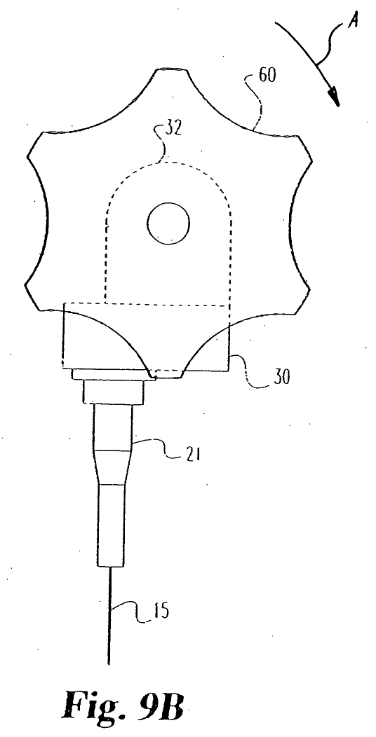

FIG. 5 is a side view of spool 50. As will be explained in greater detail below in connection with FIGS. 8A-9B, hole 52 and slot 51 receive activation line 15 from second opening 38 of base 30. Activation line 15 is coupled to hole 52 and slot 51 by slipping activation line 15 into slot 51 and placing a knot in activation line 15 into hole 52. Drive pin 40 engages spool 50, permitting spool 50 to rotate in the same direction as drive pin 40 when the drive pin 40 is rotated by knob 60. The connection between drive pin 40 and spool 50 also prevents drive pin 40 from dislodging out of second receiving opening 34. In addition, a flanged cap 41 is press fitted onto the end of drive pin 40 (opposite from knob 60) to limit movement of the drive pin 40 relative to the first end 31 of base 30. In other words, flanged cap 41 and knob 60 cooperate to prevent drive pin 40 from being completely removed from base 30.

FIGS. 6-7 illustrate the combination of activating mechanism 20 with the incorporation of a surgical snaring system 42. Surgical snaring system 42 includes snare 44, handle 45, catheter 46, flexible wire 47, operating loop 48 and electrical connector 49. Catheter 46 extends through openings 37 and 38 of second threading channel 36 (shown in FIGS. 4A-4E) of base 30. Catheter 46 also extends through openings 28 and 29 of first threading channel 27 of mounting component 21. Catheter 46, flexible wire 47 and operating loop 48 then extend through operating channel 16 of endoscope 10 and can extend through ligating barrel 14.

In the particular embodiment illustrated, surgical snaring system 42 comprises an operating loop 48 (or snare head) made from a braided stainless steel cable and having a hexagonal shape when in the open configuration. The braided stainless steel cable provides the operating loop 48 with a combination of flexibility, strength and resiliency that permits multiple resections of tissue. For example, and as will be explained in greater detail below, the ligating system 5 may comprise as many as six (or more) deployable ligating bands disposed on the ligating barrel 14. The ligating system 5 is therefore capable of banding as many as six (or more) separate tissue sections (i.e., pseudo-polyps) without withdrawing the endoscope 10 from the patient to re-load the ligating system 5 with additional ligating bands. Thus, it is preferable that a single operating loop 48 be capable of resectioning (cutting) each of the banded tissue sections (i.e., pseudo-polyps) without breaking or excessively deforming, thereby eliminating the need to withdraw and replace the original surgical snaring system 42 with a second (or third) surgical snaring system. It is also preferable that the operating loop 48 be capable of cutting through a ligating band that has been placed about the target tissue, which can occur if the operating loop 48 is not positioned completely above or below the ligating band. Similarly, the hexagonal shape of the operating loop 48 facilitates the resection of the banded tissue since the ligating bands tends to create a pseudo-poly having a generally circular cross-section that is easily ensnared by the operating loop 48. The hexagonal shape also facilitates shape retention, even after repeated use. A suitable surgical snare system 42 is the 7FR Soft AcuSnare™ Mini Hexagonal Head disposable polypectomy snare, sold by Wilson-Cook Medical Inc., dba Cook™ Endoscopy, 4900 Bethania Station Road, Winston-Salem, N.C. 27105, catalog no. SASMH-1. The 7FR Soft AcuSnare™ Mini Hexagonal Head disposable polypectomy snare has a braided stainless steel snare with a loop size of 1.5 cm×2.5 cm and a catheter sheath size of 7.0 FR. Although other types of surgical snare systems (or other types of medical catheter devices) may be utilized in combination with the ligating system 5 disclosed herein, these other devices may not be as suitable or efficient for performing multiple tissue resections.

To facilitate the extension of activation line 15 and catheter 46 all the way through first threading channel 27, first threading channel 27 and first opening 28 can be enlarged via boring from a typical diameter of about 2.5 mm to a new diameter of about 3.2 mm. A 3.2 mm diameter threading channel and first opening allows the physician to use a larger sized snare, such as the surgical snare system 42 having a 7 FR catheter sheath described above. Of course, a 3.2 mm diameter threading channel and first opening will also accommodate smaller sized snares, such as a surgical snare system having a 5FR catheter sheath. A smaller sized snare may be advantageous because it reduces frictional forces between the exterior of the snare's catheter 46, the activation line 15 of the ligating system 5, and the interior of the endoscope operating channel 16. It should also be understood that first threading channel 27 and first opening 28 can be enlarged or formed to have any diameter, within the dimensional limits of the endoscope operating channel 16 and the mounting component 21, that may be required to accommodate larger (or smaller) surgical snare systems 42 or other types of medical catheter devices there through. In the particular embodiment illustrated, the endoscope operating channel 16 has a diameter of about 3.7 mm and the mounting component 21 has an outer diameter of about 4.0 mm. Thus, the upper limit of the diameter of first threading channel 27 and first opening 28 will necessarily be less than 3.7 mm, depending on the specific design, configuration and type of materials utilized for these components. Regardless of the size of the threading channel 27 and first opening 28, it is imperative that outer sealing portion 25 forms a seal with sealing member 17 so that there is no pressure loss in the vacuum needed during endoscopic procedures.

FIGS. 8A, 8B, 9A and 9B are views of two modes of operation for activating mechanism 20. FIG. 8A depicts the disengaged mode of operation. Activation line 15 and catheter 46 are threaded through mounting component 21 and base 30 (catheter 46 is not shown in FIGS. 8A, 8B, 9A and 9B to add clarity to the components of the ligating system 5). Activation line 15 is coupled to spool 50 and ligating bands. The use of an activation line or lines to release one or more ligating bands is well known in the art and is described in U.S. Pat. No. 6,730,101, the entire contents of which are hereby incorporated herein by reference. The disengaged mode of operation is based on a disengaged position of drive pin 40 within roller clutch 70. FIG. 8A discloses drive pin 40 being substantially disposed outside of second arm 32. FIG. 8A further shows drive pin 40 being substantially disposed within first arm 31. This is the disengaged position of drive pin 40. As shown in FIG. 8B, when drive pin 40 is in the disengaged position, knob 60 can be rotated freely in either direction (A or B). After the attachment of activation line 15 to spool 50, knob 60 can be turned in either direction to wrap activation line 15 around spool 50. To unwrap activation line 15, knob 60 can be rotated in the opposite direction of the first rotation.

FIG. 9A depicts the working mode of operation. The working mode of operation is also based on a working position of drive pin 40 within roller clutch 70. FIG. 9A discloses drive pin 40 as being substantially disposed within second arm 32. FIG. 9A further shows drive pin 40 as being substantially disposed outside of first arm 31 with drive pin 40 engaging contact with roller clutch 70. As shown in FIG. 9B, when drive pin 40 is in the engaged position, knob 60 can only be rotated in one direction. After the attachment of activation line 15 to spool 50, knob 60 can be rotated in one direction to wrap activation line 15 around spool 50 under controlled tension to release one or more ligating bands from ligating barrel 14 as desired. Upon completion of the ligation procedure, activating mechanism 20 may be reset in the disengaged mode to release the tension from activation line 15 and allow activation line 15 to be unwound from spool 50 and disconnected therefrom.

FIGS. 10 and 11 show how activating mechanism 20 is mounted to endoscope 10 by inserting mounting element 21 into operating channel 16. For this illustration, endoscope 10 is shown provided with sealing member 17 which is coupled to proximal opening 18 of endoscope 10. FIG. 10 shows the alignment of activating mechanism 20 with proximal opening 18 prior to insertion of mounting element 21 therein. FIG. 11 shows mounting element 21 inserted into operating channel 16. Upon insertion, friction-fitting portion 24 friction fits with receiving portion 19 of operating channel 16, and sealing portion 25 seals with sealing member 17 of endoscope 10. And as described earlier, activation line 15 is threaded into and attached to activating mechanism 20.

Accordingly, ligating system 5 can be used in conjunction with endoscope 10 by a physician for the treatment of tissue. First, the physician positions the ligating barrel 14 (and thus the distal end of the endoscope) over the target tissue. The physician then applies suction via the endoscope to a target tissue (or pseudo-polyp) and deploys one or more ligating bands around the pseudo-polyp. Once the pseudo-polyp has been banded, the physician may release the suction and insert snaring system 42 (with a snare as large as 7 FR if the threading channel and first opening is at least 3.2 mm in diameter) through threading channel 27, operating channel 16 and ligating barrel 14. The physician can position operating loop 48 to ensnare the pseudo-polyp above the ligating band(s) with the snaring system. Next, the physician can cut the pseudo-polyp through electric cautery via electrical connector 49. The resected piece of tissue (i.e., pseudo-polyp) is then typically allowed to drop into the esophagus and/or the stomach, where it may pass naturally through the digestive tract of the patient. If desired, the physician may retrieve the resected pieces of tissue with a separate forceps (not shown), the operating loop 48 of the snaring system 42, or some other tissue collecting device. Retrieval of the resected pieces of tissue is preferably done after all of the resections have been completed. In particular, the present invention permits the physician to perform multiple band ligation and Endoscopic Mucosal Resection (EMR) in the esophagus and other portions of the gastrointestinal tract. This procedure can be repeated several times during one surgical session, increasing the physician's efficiency.

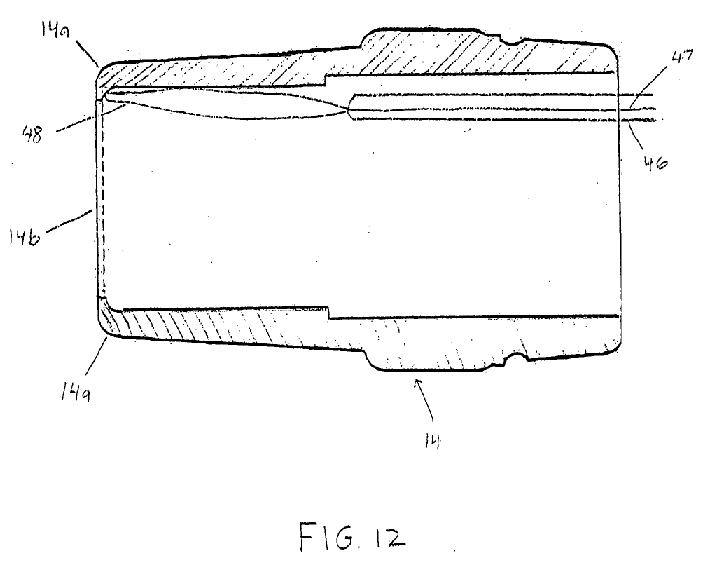

Alternatively, the physician can insert snaring system 42 through the first threading channel 27 and operating channel 16 at the beginning of the procedure, before applying suction and banding the pseudo-polyp. In this method, the physician can guide operating loop 48 all the way through operating channel 16 into ligating barrel 14. As illustrated in FIG. 12, the physician can then push operating loop 48 up against the interior surface of the distal rim 14a of the ligating barrel, just inside of the distal opening 14b of the barrel. This orientation serves to minimize the degree to which elements of the snaring system may interfere with the suction and banding steps by the physician. Although the particular embodiment shown in FIG. 12 illustrates the distal rim 14a as having an inwardly projecting flange against which the operating loop 48 is secured, the distal rim 14a may alternatively comprise a smooth, non-flanged interior surface. A non-flanged interior surface is less likely to interfere with extension, manipulation, and retraction of the operating loop 48. In order to further minimize this potential interference, a smaller sized snare (5 FR) can be used, which also permits the use of a smaller diameter (about 2.5 mm) for the first threading channel 27 and first opening 28.

Moreover, additional endoscopic devices and procedures can be combined with the activating mechanism of the ligating system due to the increased diameter in threading channel 27 and first opening 28. Thus, an injection needle for sclerotherapy or an endoscopic ultrasound (EUS) needle could be extended through threading channel 27 and first opening 28 into and through the operating channel 16 of endoscope 10. In addition, biopsy forceps could also be utilized (instead of suction) by extending the forceps through the threading channel to capture the tissue and withdraw the tissue into the ligating barrel before deploying ligating bands. These other medical instruments can be utilized to treat the target tissue either before or after the banding of the tissue. Furthermore, these instruments can be used serially in one surgical session. For example, a solution can first be injected into the submucosal layer of tissue to elevate the target tissue and separate it from the muscular layer before banding the target tissue with a ligating system and removing it with a snaring system. It should be noted that the present invention can also be used to treat vessels as well as tissue.

It will of course be well understood from the discussions above that other known ligating barrel designs, activation mechanisms, endoscope systems, etc. could be used within the scope of the invention. It is therefore intended that the foregoing detailed description be regarded as illustrative rather than limiting, and that it be understood that it is the following claims, including all equivalents, that are intended to define the spirit and scope of this invention.

1. A system for endoscopic treatment of tissue, the system comprising:
a ligating system comprising an activating component and a ligating barrel operably connected to the activating component, the activating component being adapted to be connected to a proximal end of an endoscope operating channel, the ligating barrel being adapted to be disposed on a distal end of the endoscope; and
a medical instrument comprising an elongate shaft and an operating member disposed near a distal end of the elongate shaft, the elongate shaft being adapted to be simultaneously disposed through the activating component, the endoscope operating channel, and the ligating barrel so as to position the operating member near the tissue to be treated.

2. The system of claim 1 wherein the ligating system further comprises an activation line operably connected between the activating component and the ligating barrel, the activation line being adapted to extend through the endoscope operating channel.

3. The system of claim 2 wherein the activation line and the elongate shaft are adapted to be simultaneously of the disposed through the endoscope operating channel in a side-by-side configuration

4. The system of claim 2 further comprising one or more ligating bands removably disposed on the ligating barrel and coupled to the activating component via the activation line, wherein operation of the activating component releases one or more ligating bands from the ligating barrel.

5. The system of claim 1 wherein the operating member of the medical instrument is an electrosurgical snare.

6. A system for endoscopic treatment of tissue, the system comprising:
an endoscope having a proximal end, a distal end, and an operating channel extending between a proximal opening and a distal opening;
a ligating system comprising an activating component and a ligating barrel, the activating component having a mounting component with a first threading channel, the mounting component being connected to the proximal opening of the operating channel of the endoscope, the ligating barrel being disposed on the distal end of the endoscope and being operably connected to the activating component; and
a medical instrument comprising an elongate shaft and an operating member disposed near a distal end of the elongate shaft, the elongate shaft being disposed through the first threading channel, the operating channel, and the ligating barrel so as to position the operating member near the tissue to be treated.

7. The system of claim 6 wherein the ligating system further comprises an activation line extending through the operating channel of the endoscope and operably connected between the activating component and the ligating barrel.

8. The system of claim 7 further comprising one or more ligating bands removably disposed on the ligating barrel and coupled to the activating component via the activation line, wherein operation of the activating component releases one or more ligating bands from the ligating barrel.

9. The system of claim 6 wherein the threading channel of the mounting component has a diameter of about 2.5-3.2 mm.

10. The system of claim 6 wherein the mounting component further comprises a coupling portion, a friction-fit portion, and an outer sealing portion.

11. The system of claim 6 wherein the activating component comprises a base, a drive pin, a spool, a knob, and a roller clutch.

12. The system of claim 6 wherein the operating member of the medical instrument is an electrosurgical snare.

13. The system of claim 12 wherein the snare comprises a loop having a hexagonal shape.

14. The system of claim 12 wherein the snare comprises a braided loop.

15. The system of claim 7 wherein the braided loop is formed from a braided stainless steel cable.

16. The system of claim 12 wherein the elongate shaft of the medical instrument has an exterior diameter of about 5-7 FR.

17. The system of claim 6 wherein the mounting component is formed of plastic or polyurethane.

18. The system of claim 6 wherein the mounting component is formed of aluminum.

19. A method for endoscopic treatment of tissue, the method comprising the steps of:
providing an endoscope having a proximal end, a distal end, and an operating channel extending between a proximal opening and a distal opening having an operating channel;
providing a ligating system comprising an activating component, an activation line, and a ligating barrel;
providing a medical instrument comprising an elongate shaft and an operating member disposed near a distal end of the elongate shaft,
disposing the ligating barrel on the distal end of the endoscope, connecting the activating component to the proximal opening of the operating channel, disposing the activation line through the operating channel, and operably connecting the activation line between the ligating barrel and the activating component;
disposing the shaft of the medical instrument through the activation component, the operating channel, and the ligating barrel;
positioning the ligating barrel over target tissue, wherein the ligating barrel carries one or more ligating bands operably connected to the activation line;
drawing tissue into the ligating barrel;
deploying one or more ligating bands around the target tissue;
releasing the target tissue from the ligating barrel; and
engaging the target tissue with the operating member of the medical instrument.

20. The method of claim 19 wherein the tissue is drawn into the ligating barrel with suction.

21. The method of claim 19 wherein operation of the activating component pulls the activation line proximally to release one or more ligating bands from the ligating barrel.

22. The method of claim 19 wherein the operating member of the medical instrument is an electrosurgical snare, and the step of engaging the target tissue comprises the step of electrosurgically resectioning the target tissue.

23. The method of claim 22 wherein the snare comprises a loop having a hexagonal shape.

24. The method of claim 22 wherein the snare comprises a braided loop.

25. The method of claim 24 wherein the braided loop is formed from a braided stainless steel cable.

26. The method of claim 22 wherein the elongate shaft of the medical instrument has an exterior diameter of about 5-7 FR.
